(19) United States
(12) Patent Application Publication
(10) Pub. No.: US 2017/0354369 A1
(43) Pub. Date: Dec. 14, 2017

(54) METHODS AND SYSTEMS FOR TESTING OPTICOKINETIC NYSTAGMUS

(71) Applicant: THE TRUSTEES OF THE UNIVERSITY OF PENNSYLVANIA, Philadelphia, PA (US)

(72) Inventor: Monte Mills, Philadelphia, PA (US)

(21) Appl. No.: 15/527,532

(22) PCT Filed: Nov. 20, 2015
(86) PCT No.: PCT/US15/61900

§ 371 Date: May 17, 2017

Related U.S. Application Data
(60) Provisional application No. 62/082,226, filed on Nov. 20, 2014.

(51) Int. Cl. A61B 5/00; A61B 3/00; A61B 5/11; A61B 3/113

(52) U.S. Cl. CPC A61B 5/4863; A61B 3/113; A61B 5/7425; A61B 5/744; A61B 3/0058; A61B 5/7282; A61B 5/0077; A61B 5/1128; A61B 3/0091; A61B 3/0025; A61B 5/6898

(57) ABSTRACT

A mobile system for measuring opticokinetic nystagmus in a subject includes a display screen to provide an opticokinetic stimulus and an imaging system to record eye movement data of the subject. The mobile system is configured to compare the stimulus and the recorded eye movement data to provide objective vision acuity testing.

### CROSS-REFERENCE TO RELATED APPLICATIONS

This application claims priority to U.S. Provisional Application Ser. No. 62/082,226 filed on Nov. 20, 2014, which is incorporated by reference herein in its entirety.

### BACKGROUND

Opticokinetic nystagmus (OKN) refers to reflexive (i.e., involuntary) eye movement elicited in response to moving targets. A subject's OKN response can be tested by providing the subject with a stimulus and measuring eye movement, which provides certain information concerning the subject's vision.

Certain existing OKN test systems are qualitative in nature and do not provide quantitative measure of a subject's visual acuity. Other OKN test methods involve complex and bulky apparatus and have not become generally established for routine testing. There exists a need in the field of OKN testing to develop a mobile testing system.

### SUMMARY

The presently disclosed subject matter provides methods and systems for testing OKN. In certain embodiments, the methods and systems for testing OKN can include a mobile platform with a display screen and a camera.

In certain embodiments, methods of measuring OKN in a subject are provided. An exemplary method can include generating at least one moving stimulus having a least a first parameter. The first parameter can include a direction of motion, a rate of motion, a change in dimensions, and a change in colors. The method can further include displaying the stimulus on a display screen of a mobile device. The method can include detecting, using an imaging system, eye data having at least a second parameter obtained from the subject viewing the stimulus. The second parameter can include eye movement velocity, eye movement direction, and a combination thereof. In certain embodiments, the method can include comparing the first parameter and second parameter.

In certain embodiments, the method can include varying the first parameter over time. Varying the first parameter over time can include crossing a threshold of perceptibility of the subject. The moving stimulus can include a moving pattern. In certain embodiments, the moving pattern can be a grating pattern. In certain embodiments, the moving pattern can provide a constant luminance on the display screen. In certain embodiments, the constant luminance can be about 10 candelas/m². In certain embodiments, the velocity of the moving pattern can be about 10 degree per second. In certain embodiments, the method can include diagnosing a visual acuity of the subject. In certain embodiments, the method can include detecting abnormal eye movements.

In certain embodiments, the disclosed subject matter relates to a system for measuring OKN in a subject. In certain embodiments, the system can include a display screen, an imaging system, one or more processors, and a memory. In certain embodiments, the imaging system can be configured to detect eye data. In certain embodiments, the processors can be functionally coupled to the display screen and the imaging system. In certain embodiments, the memory can be coupled to the processors. In certain embodiments, the processors can be operable when executing the instructions to generate at least one moving stimulus having at least a first parameter, display the stimulus on the display screen, receiving from the imaging system the eye data having at least a second parameter, and comparing the first parameter and second parameter. In certain embodiments, the processors are in communication with a network device for comparing the first parameter and the second parameter. In certain embodiments, the imaging system can include a front-facing camera of a tablet.

### BRIEF DESCRIPTION OF THE DRAWINGS

FIG. 1 provides a diagram of an exemplary mobile device in accordance with the disclosed subject matter.

FIG. 2 provides a flow chart of an exemplary method in accordance with the disclosed subject matter.

FIG. 3 provides exemplary patterns as displayed on mobile devices.

FIG. 4 provides a flow chart of an exemplary method of conducting OKN test.

FIG. 5 illustrates a typical OKN response showing eye position, in degrees, over time.

FIG. 6 is exemplary image capture of a side-by-side composite video.

FIG. 7 illustrates deploying the disclosed subject matter in network environments.

FIG. 8 provides example eye movement data captured in a 3-second test period, suggesting good tracking response.

FIG. 9 provides example eye movement data captured in a 3-second test period, suggesting fixed staring.

FIG. 10 provides example eye movement data captured in a 3-second test period, when the left eye is covered.

### DESCRIPTION

The disclosed subject matter provides methods and systems for testing OKN. For example, the methods and systems can use a mobile platform with a display screen and a camera. In certain embodiments, the system can link and automate an opticokinetic stimulus on the screen and detect eye movements with the camera. This can allow for objective vision testing. In addition to testing vision, in certain embodiments, the methods and systems disclosed herein can measure abnormal eye movements and alignment, for example, strabismus, pathological nystagmus, and other normal and abnormal visual-motor conditions.

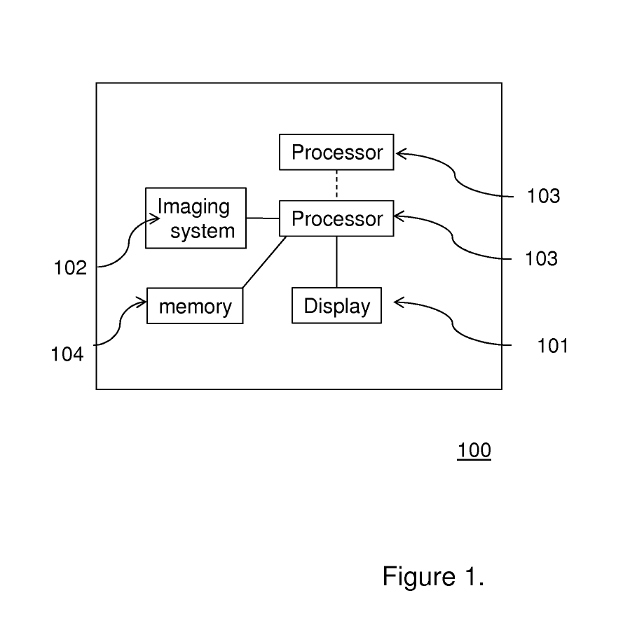

For the purpose of illustration and not limitation, FIG. 1 is a diagram of an exemplary system. In certain embodiments, a mobile device 100 can include a display screen 101 and an imaging system 102 (for example, a camera). The display screen 101 and imaging system 102 can be linked to the same mobile device. The mobile device can be, for example, an IPad, IPhone, Surface tablet, laptop computer, or other suitable mobile computing device. In certain embodiments, the system 100 can include a separate imaging system 102. In certain embodiments, the mobile device can include more than one mobile computing devices, e.g. two IPads. In certain embodiments, the mobile device can include one or more display screens, e.g. one or more IPads.

In certain embodiments, the system can include a display screen 101, an imaging system 102, one or more processors 103, and a memory 104. In certain embodiments, the imaging system 102 can be configured to detect eye data. In certain embodiments, the processors 103 can be coupled to the display screen 101 and the imaging system 102. In certain embodiments, the memory 104 can be coupled to the processors 103. In certain embodiments, the processors 103 can be operable when executing the instructions to generate at least one moving stimulus having at least a first parameter, display the stimulus on the display screen 101, receiving from the imaging system 102 the eye data having at least a second parameter, and comparing the first parameter and second parameter.

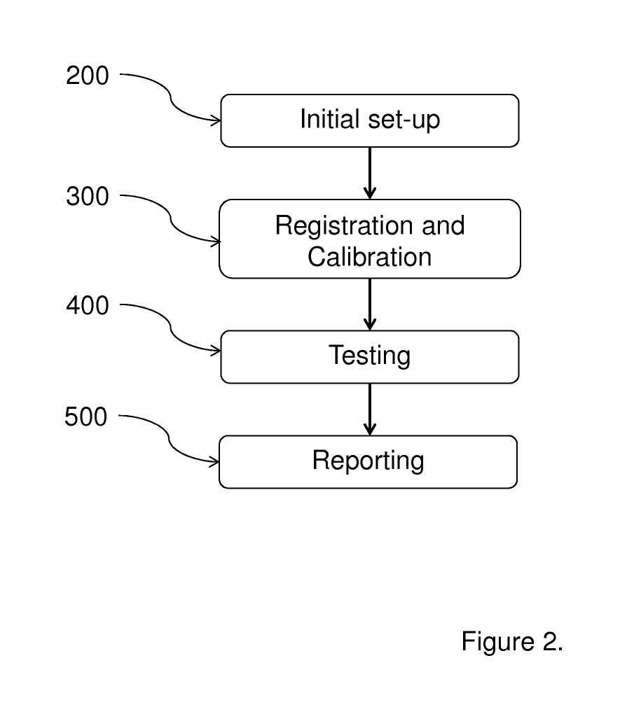

For the purpose of illustration and not limitation, FIG. 2 is a flow chart of an exemplary method in accordance with the disclosed subject matter. In certain embodiments, the disclosed subject matter can include initial set-up 200. Setup data can be entered into the system. Such data can include subject demographics, physical data including interpupillary distance, and other condition including known/suspected eye conditions and comparative vision information. In certain embodiments, the mobile device can be oriented in a “reverse portrait” position, whereby a front-facing camera is located below the display screen. In certain embodiments, the imaging system can allow testing at a variety of testing distances and without a fixed head portion (freely moving head). In certain embodiments, the testing distance can be fixed to from about 30 to 60 cm, e.g. 38 cm or 55 cm. For certain embodiments, the distance can be calculated based on the captured image and other data such as the known interpupillary distance of the subject.

In certain embodiments, the disclosed subject matter further includes registration and calibration 300. In certain embodiments, appropriate attention animations can be displayed to attract the attention of the subject. For example, large face cartoons can be compelling for early infancy. For infants older than 6 months, starburst and geometric animations can be effective. For children older than 1, animations of animals or people can be effective.

In certain embodiments, the camera can be turned on for an initial registration, including capture images of stable eye alignment for automatic tests.

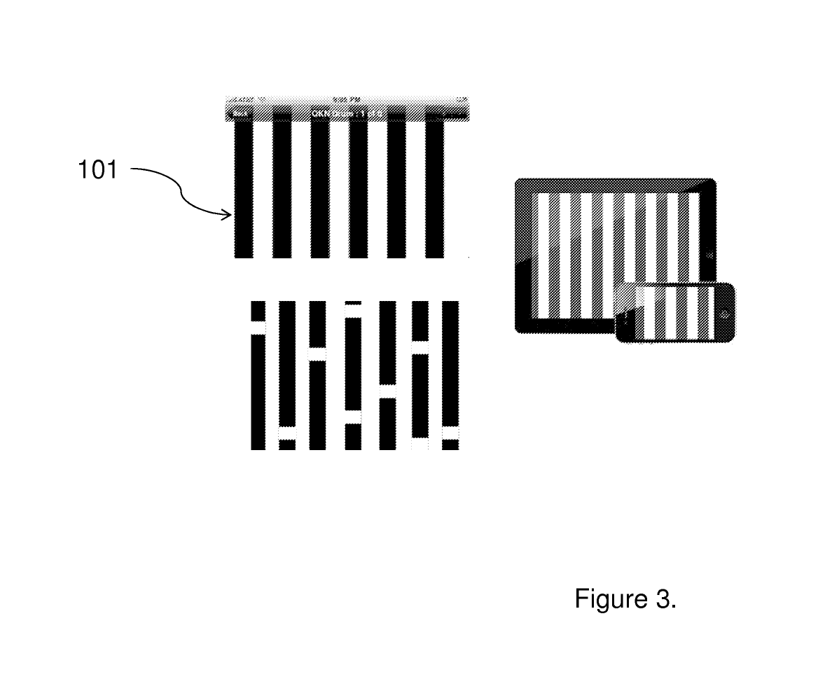

In certain embodiments, the disclosed subject matter further includes a testing phase 400. In certain embodiments, a stimulus can be displayed on the display screen. The stimulus can be, for example, gratings or other patterns which are moving and/or scalable. For the purpose of illustration and not limitation, FIG. 3 demonstrates certain gratings as displayed on mobile devises. In certain embodiments, the display screen has constant luminance, i.e., the net brightness of the whole screen remains stable during the testing. In certain embodiments, the display screen can have constant luminance of from about 8 to about 12 candelas/m², e.g. about 10 candelas/m².

The stimulus can include various combinations of motion (e.g., horizontal, vertical, left, right, or other suitable motions), variable rate of movement, variable pattern dimensions (e.g., large-small), and the capacity to change the pattern dimensions (e.g., size, speed, direction) during the course of the test. Other components of the stimulus image (e.g., contrast, color, or pattern) can also be modified depending on the situation and testing needed. In certain embodiments, the moving stimulus can be calibrated to maintain a velocity of 10 degrees per second at all testing distances. In certain embodiments, the velocity of the moving stimulus can be reduced to avoid blurring that can potentially be caused by the limited display performance of certain mobile devices. Direction can matter in certain types of patients and tests. In certain embodiments, the moving stimulus can have a default horizontal direction of movement. In certain embodiments, the moving stimulus can have a vertical movement. Certain patient can have directional asymmetry, for example, higher acuity with temporal-to-nasal direction than nasal-to-temporal.

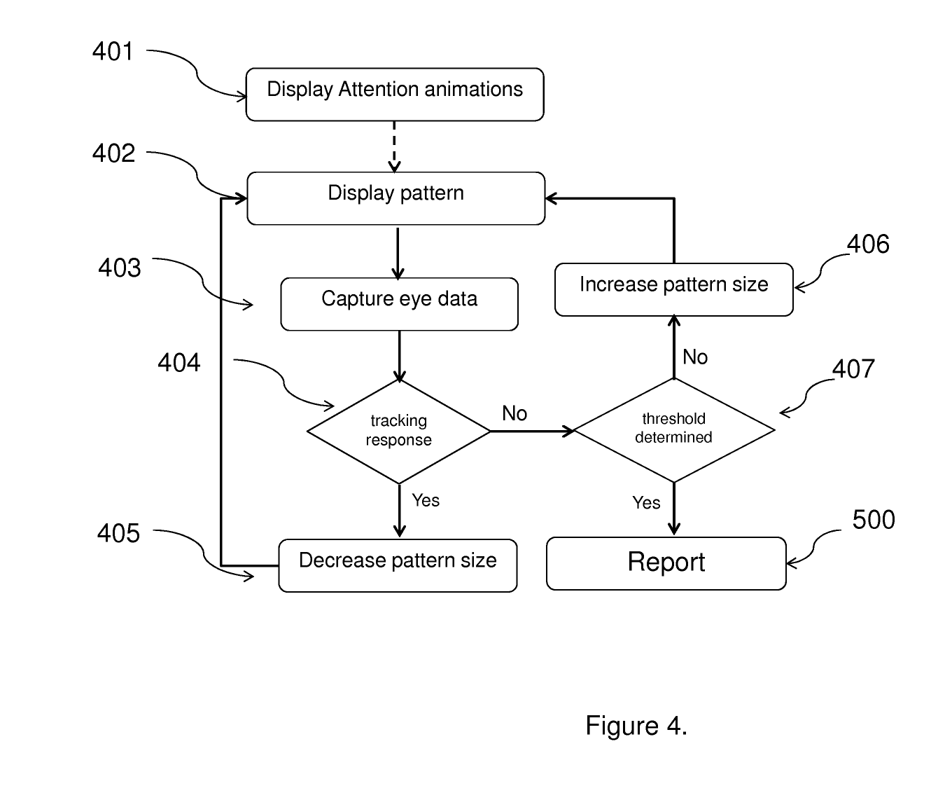

For the purpose of illustration and not limitation, FIG. 4 is a flow chart of an exemplary method of testing 400. After an initial display of attention animations 401 appropriate for the age of the subject, the stimulus can, for example, start with a coarse large pattern 402. In certain embodiments, the size of the initial patterns, for example, stripes, can be scalable to allow testing at a pattern size large enough to be easily seen by the subject (above the acuity threshold) at the testing distance. The pattern can be calibrated such that the size of the pattern or other parameters of the pattern (e.g., cycles per degree (cpd), log MAR, Snellen equivalent) can be adjusted based on, for example, the situation, screen size, testing distance, etc. Table 1 shows an exemplary correlation between numeric values of cpd, log MAR, and Snellen equivalent. In certain embodiments, the size of the initial patterns can depend on the age of the subject. For example, the initial patterns can have cpd of 0.2 for infant less than 6 months old and cpd of 0.8 for older infants and children.

TABLE 1
Correlation between numeric values of
cpd, logMAR, and Snellen equivalent.

| Snellen equivalent | LogMAR | Cycles per degree (cpd) |
|---|---|---|
| 6/12 (20/40) | 0.3 | 15.0 |
| 6/18 (20/60) | 0.5 | 10.0 |
| 6/24 (20/80) | 0.6 | 7.5 |
| 6/36 (20/120) | 0.8 | 5.0 |
| 6/48 (20/160) | 0.9 | 3.75 |
| 6/60 (20/200) | 1.0 | 3.0 |
| 6/72 (20/240) | 1.1 | 2.5 |
| 6/90 (20/300) | 1.2 | 2.0 |
| 6/120 (20/400) | 1.3 | 1.5 |
| 6/150 (20/500) | 1.4 | 1.2 |
| 6/180 (20/600) | 1.5 | 1.0 |
| 6/240 (20/800) | 1.6 | 0.75 |
| 6/360 (20/1200) | 1.8 | 0.50 |
| 6/480 (20/1600) | 1.9 | 0.28 |

In certain embodiments, the imaging system (e.g., camera or eye position detector) can be used to capture information 403 regarding eye movements or “tracking behavior” during the stimulus test. In certain embodiments, the system can include a software configured to extract the parameters of the eye movement from the captured images. In certain embodiments, the system can use movements of the corneal light reflection (Purkinje images) or other components of the images of the eyes produced by the imaging system. In certain embodiments, the system can compare the parameters of the eye movements detected, for example, OKN, including direction and speed, to the stimulus parameters. In certain embodiments, the parameter of the eye movement detected can be compared to the stimulus to determine whether the moving stimulus is able to elicit a tracking response 404 from the test subject.

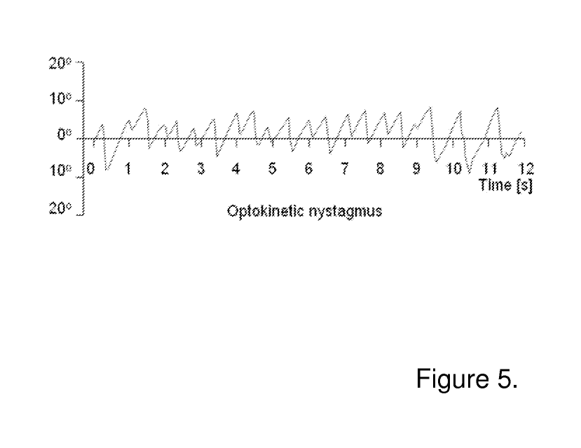

For example, FIG. 5 illustrates a typical OKN response showing eye position, in degrees, over time. The OKN response has a slower “smooth pursuit” phase, in the direction of the target, and a fast “saccadic” or recovery phase, in the opposite direction.

In certain embodiments, even in the absence of an imaging system, the stimulus can be used by an observer (e.g., through direct observation of the subject's response) to measure visual acuity or perform other eye tests. In certain embodiments, the image of the eye movement captured by the imaging system can be composited with the stimulus presented for a side-by-side comparison on a common timeline.

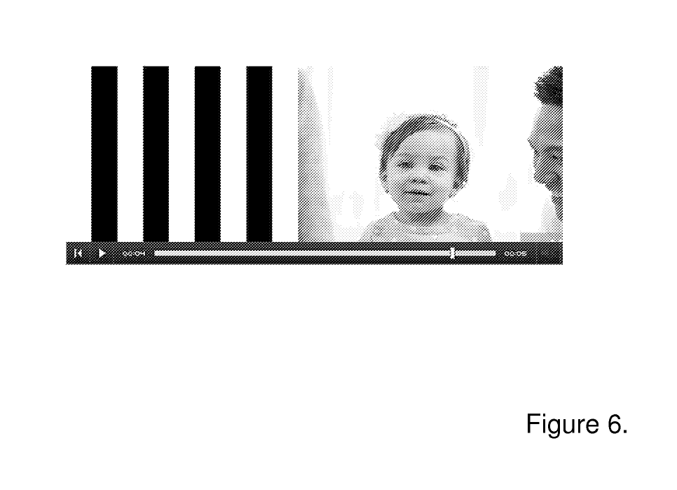

For the purpose of illustration and not limitation, FIG. 6 shows an exemplary image capture of a side-by-side composite video. Such side-by-side composite video can be provided to a doctor for assessment or become a part of patent record. In certain embodiments, system can be connected to network for remote data analysis, proceeding, and storage.

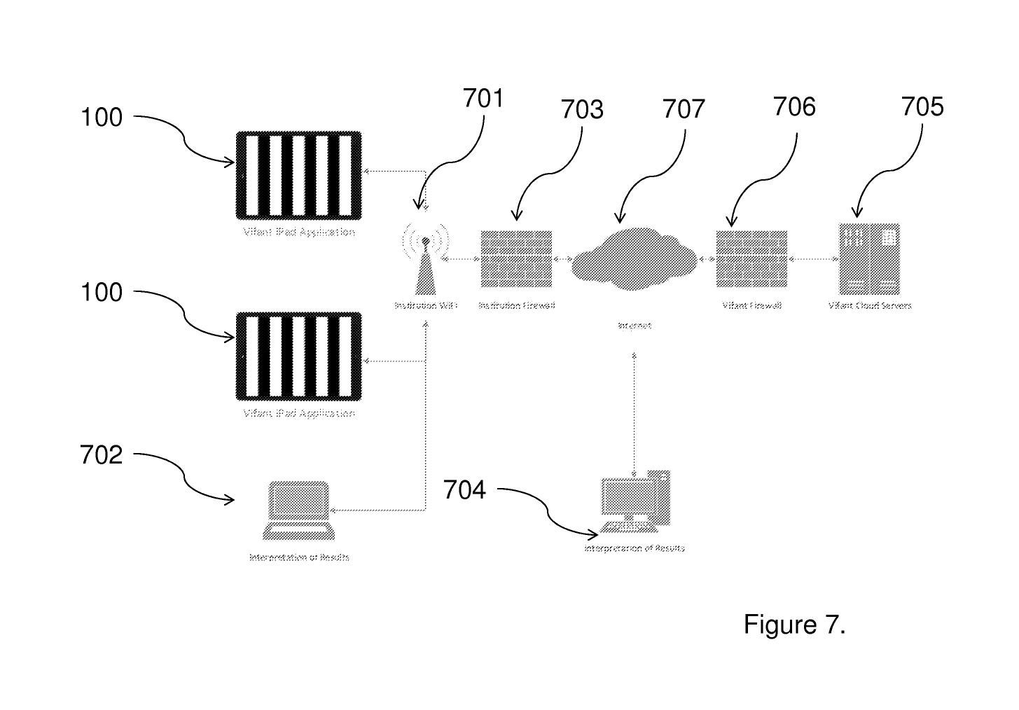

For example, FIG. 7 illustrates means to deploy the disclosed subject matter over a networking environment. Multiple mobile devices 100 can be distributed and connected to a local area network 701 and data generated by the mobile devices can be analyzed or interpreted with a local workstation 702. In certain embodiments, the data can be passed through firewall 703 and transmitted to a workstation 704 connected to the internet. In certain embodiments, the data be stored in secure cloud servers 705 protected by a firewall 706. Necessary data security measurement can be in place to ensure compliance with HIPPA and other regulatory requirements.

In certain embodiments, the system can include a software configured to compare the detected eye movement data to the stimulus. In certain embodiments, the system can extract the slow phase smooth pursuit velocity to compare with the velocity of the moving stimulus. In certain embodiments, slow phase smooth pursuit velocity within 30% of pattern velocity for a certain proportion of the test period (e.g. 4 seconds of a 20 second testing period) can be considered successful tracking response. In certain embodiments, the system can extract the slow phase smooth pursuit direction and compare that with the direction of the moving stimulus. In certain embodiments, slow phase smooth pursuit direction within 45 degrees of pattern direction for a threshold fraction of the test period can be considered successfully tracking response. In certain embodiments, both the slow phase smooth pursuit direction and velocity can be combined to determine a successful tracking response. In certain embodiments, a mathematic model can be constructed to evaluate the tracking response.

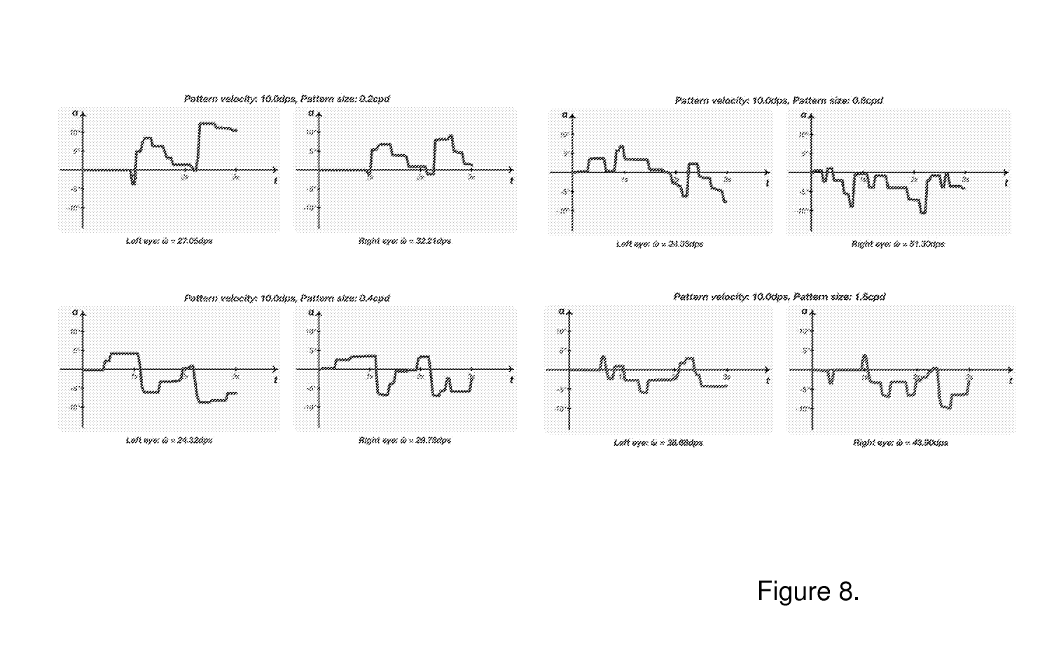

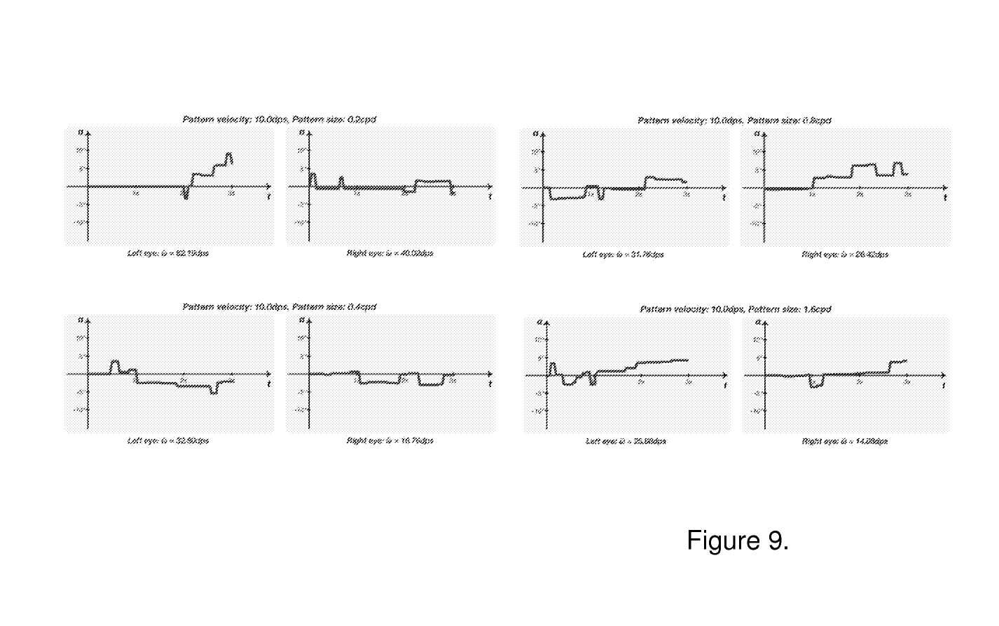

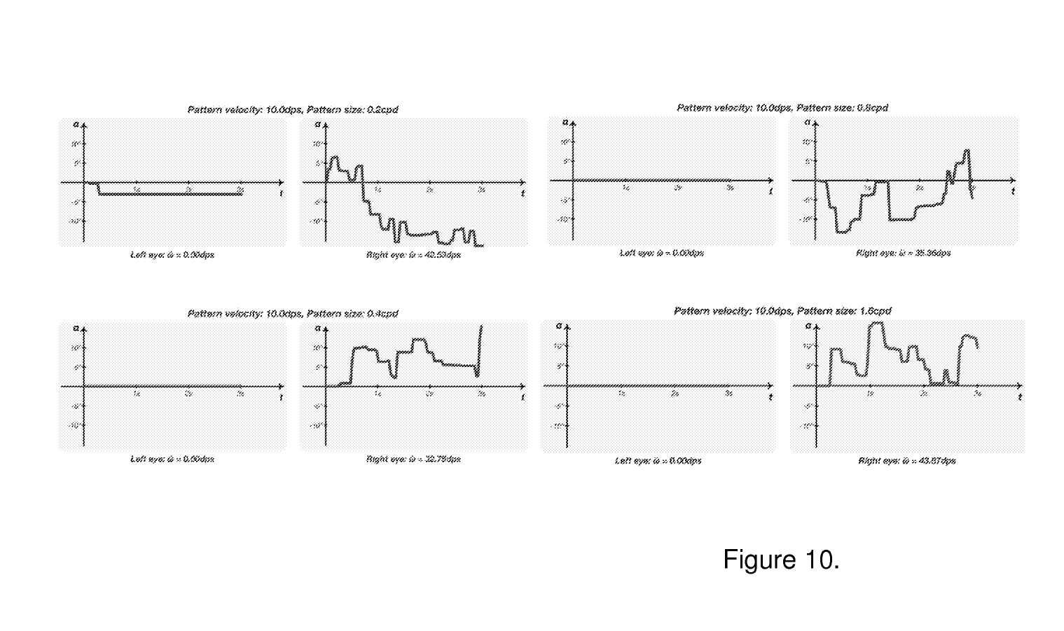

For the purpose of illustration and not limitation, FIG. 8 shows example eye movement data captured in a 3-second test period, showing good tracking response. FIG. 9 shows example eye movement data captured in a 3-second test period, suggesting fixed staring. In certain embodiments, monocular occlusion, detecting motion with only one eye exposed, can be performed. FIG. 10 shows example eye movement data captured in a 3-second test period, when the left eye is covered. In certain embodiments, the system is configured to detect lost attention, i.e. eyes off field. In certain embodiments, an examiner can note lost attention. The display can be reverted to attention patterns 401. The lost attention period can be excluded from the analysis of the OKN response.

In certain embodiments, for example, for visual acuity testing, the system can be configure to sweep the pattern size from larger to smaller during test until the pattern size becomes too smaller for the subject to follow. The smallest patterns can be at 30 cpd or less (Snellen equivalent 20/20). In certain embodiments, where the eye movements stop tracking the pattern movement can be recognized as the visual threshold resolution acuity (visual acuity). In certain embodiments, pattern size can be increased to sweep across and re-cross the visual threshold repeatedly until the visual acuity threshold is consistently determined 407. In certain embodiments, movement direction of the stimulus can be reversed between each test period or between change of pattern sizes. With each change in direction, there can be a lag between the pattern change and the eye movement change. This latency can vary with age and other test conditions, such as monocular or binocular. In certain embodiments, this latency period can be excluded from the analysis of tracking response. In certain embodiments, repetitive testing can increase the precision and reliability of the measurement.

In certain embodiments, the report 500 can be in any form deemed useful for a health service profession or a research. In certain embodiments, the report 500 can be in the form of a side-by-side composite video for further analysis. In certain embodiments, the report 500 can be in a numeric value of the visual acuity determined according to above disclosed procedure. In certain embodiments, the report 500 can be a pass or fail based on the determination of visual acuity, according to the age dependent testing threshold, for example, in Table 2.

TABLE 2
Estimated Testing Thresholds by Age,
OKN Test, 95% CL, Lower threshold
threshold, CPD

| age, | | | intraocular |
|---|---|---|---|
| months | monocular | binocular | difference |
| 2 | 0.5 | 0.5 | n/a |
| 4 | 1 | 1 | 0.5 |
| 6 | 1.5 | 2 | 0.7 |
| 12 | 1.8 | 2.2 | 0.9 |
| 18 | 3.5 | 4.3 | 1.2 |
| 24 | 5 | 7.4 | 2.5 |
| 30 | 10 | 12 | 5 |
| 36 | 15 | 16 | 7.5 |
| 48 | 18 | 18 | 9 |

In certain embodiments, the disclosed subject matter can allow testing visual acuity of non-verbal subjects, for example, babies and children too young to perform recognition vision tests. Furthermore, in certain embodiments, the disclosed subject matter can be used without requiring understanding or conscious response from the subject, and does not require literacy, recognition, language, or other subjective interpretation. Additionally, in certain embodiments, the disclosed subject matter can also be used for testing uncooperative or inconsistent subject, or for testing without relying upon recognition and response, because the OKN reflex is involuntary and automatic. In certain embodiments, the disclosed subject matter can be adapted for additional types of vision testing, for example, for use with animals. In certain embodiments, the disclosed subject matter can be used for testing other components of visual testing, including color vision (e.g., by shifting color rather than pattern size), contrast sensitivity, or other components of vision. In certain embodiments, the disclosed subject matter can be used to detect and measure abnormal eye movements including strabismus, pathological nystagmus, and other eye movement disorders. In certain embodiments, the measurements can be made in “real space” without optical manipulation or lenses, and can be adapted to testing under a variety of optical conditions (e.g., ambient light, glasses, testing distances, etc.). The disclosed subject matter can allow for testing anywhere.

In certain embodiments, observation of eye movements in response to moving targets (OKN) can be used for testing for acute concussion, intoxication, or other neurological tests. For example, the disclosed subject matter can be used by law enforcement or others for testing for acute intoxication, doctors or trainers for testing athletes (or others) for acute concussion syndromes, and in civilian and military uses to test for alertness and visual-motor response before, during, and after various tasks.

In certain embodiments, the disclosed subject matter can be used by professionals interested in testing vision in babies and young children, for example, ophthalmologists, optometrists, pediatricians, parents, vision researchers, therapists, teachers, and school nurses, and other people who cannot perform other types of vision testing. In certain embodiments, the disclosed subject matter can be used by those interested in detecting abnormal eye movements and tracking behaviors, for example, sports coaches, trainers, parents, law enforcement, neurologists, pediatricians, ophthalmologist, military personnel. In certain embodiments, the disclosed subject matter can be used by those requiring visual acuity to perform certain tasks (e.g. drivers, pilots, and people on night shifts). In certain embodiments, the disclosed subject matter can be used by people interested in animal vision (e.g., veterinarians, pet owners, animal trainers, and animal researchers), and by people interested in other aspects of visual function including color or contrast vision (e.g., vision researchers, ophthalmologists, and optometrists).

In certain embodiments, the disclosed subject matter can be used for therapeutic intervention. For example, but not by way of limitation, the system can provide feedback to the subject, which can train the subject for improved visual tracking.

The description herein merely illustrates the principles of the disclosed subject matter. Various modification and alterations to the described embodiments will be apparent to those skilled in the art in view of the teachings herein. Accordingly, the disclosure herein is intended to be illustrative, but not limiting, of the scope of the disclosed subject matter.

1. A method of measuring opticokinetic nystagmus (OKN) in a subject, comprising:
generating at least a stimulus having at least a first parameter;
displaying the stimulus on a display screen of a mobile device;
detecting, using an imaging system, eye data having at least a second parameter obtained from the subject viewing the stimulus; and
comparing the first parameter and the second parameter.

2. The method of claim 1, further comprising varying the first parameter over time.

3. The method of claim 2, wherein varying the first parameter over time comprises crossing a threshold of perceptibility of the subject.

4. The method of claim 1, wherein the stimulus comprises a moving pattern.

5. The method of claim 4, wherein the moving pattern comprises a grating pattern.

6. The method of claim 4, wherein the first parameter is selected from the group consisting of a direction of motion, a rate of movement, a change in dimensions, a change in colors, and any combinations thereof.

7. The method of claim 4, wherein the display screen shows a constant luminance while displaying the moving pattern.

8. The method of claim 4, wherein the display screen shows a constant luminance of about 10 candelas/m² while displaying the moving pattern.

9. The method of claim 1, wherein distance between the screen and the subject is about 30 to 60 cm.

10. The method of claim 1, wherein distance between the screen and the subject is about 55 cm.

11. The method of claim 1, wherein the stimulus comprises an attention animation.

12. The method of claim 1, wherein the mobile device comprises a tablet computer with a front-facing camera.

13. The method of claim 12, wherein the imaging system comprises the front-facing camera.

14. The method of claim 4, wherein the second parameter is selected from the group consisting of eye movement velocity, eye movement direction, and a combination thereof.

15. The method of claim 14, wherein the velocity of the moving pattern is about 10 degree per second.

16. The method of claim 1, wherein the comparing comprises a side-by-size video comparison between the stimulus and the eye data.

17. The method of claim 1, further comprising diagnosing a visual acuity of the subject.

18. The method of claim 1, further comprising detecting abnormal eye movements.

19. A mobile system for measuring opticokinetic nystagmus (OKN) in a subject, comprising:
a display screen;
an imaging system, configured to detect eye data having at least a second parameter;
one or more processors functionally coupled to the display screen and imaging system; and
a memory coupled to the processors, the one or more processors operable when executing instructions to:
generate at least a stimulus having at least a first parameter;
display the stimulus on the display screen;
receive from the imaging system the eye data; and
compare the first parameter and second parameter.

20. The mobile system of claim 19, wherein the moving stimulus comprises a moving pattern.

21. The mobile system of claim 20, wherein the moving pattern comprises a grating pattern.

22. The mobile system of claim 19, wherein the first parameter is selected from the group consisting of a direction of motion, a rate of movement, a change in dimensions, a change in color, and any combinations thereof.

23. The mobile system of claim 19, wherein the second parameter is selected from the group consisting of an eye movement velocity, an eye movement direction, and a combination thereof.

24. The mobile system of claim 19, wherein the display screen shows a constant luminance while displaying the moving pattern.

25. The mobile system of claim 19, wherein the display screen shows a constant luminance of about 10 candelas/m² while displaying the moving pattern.

26. The mobile system of claim 19, wherein the stimulus comprises an attention animation.

27. The mobile system of claim 19, wherein the one or more processors are in communication with a network device for comparing the first parameter and the second parameter.

28. The mobile system of claim 19, wherein the imaging system comprises a front-facing camera of a tablet.
